(19) United States
(12) Patent Application Publication
(10) Pub. No.: US 2013/0144120 A1
(43) Pub. Date: Jun. 6, 2013

(54) SOLID-STATE IMAGING DEVICE AND ENDOSCOPE DEVICE

(71) Applicant: OLYMPUS CORPORATION, Tokyo (JP)

(72) Inventor: Susumu Yamazaki, Tokyo (JP)

(73) Assignee: OLYMPUS CORPORATION, Tokyo (JP)

(21) Appl. No.: 13/655,032

(22) Filed: Oct. 18, 2012

(30) Foreign Application Priority Data
Oct. 18, 2011 (JP) 2011-228481

(51) Int. Cl. H01L 27/146; A61B 1/06

(52) U.S. Cl. CPC H01L 27/14643; A61B 1/06
USPC 600/109; 250/208.1

(57) ABSTRACT

A solid-state imaging device and an endoscope device can correct characteristics of a digital signal output from an AD conversion unit with respect to a pixel signal input to an AD conversion unit with higher precision even when a dynamic range of a pixel signal is changed. A correction unit corrects the digital signal output from the AD conversion unit based on a correction function using the digital signal output from the AD conversion as a variable so as to correct the characteristics of the digital signal output from the AD conversion unit with respect to the pixel signal input to the AD conversion unit. A correction method changing unit changes an order of a variable between first and other orders in the correction function according to a change in a dynamic range of the pixel signal.

### BACKGROUND OF THE INVENTION

1. Field of the Invention

The present invention relates to a solid-state imaging device including an analog to digital (hereinafter referred to as ‘AD’) conversion unit that converts an analog voltage signal output from a pixel into a binary digital data and an endoscope device.

This application claims priority to and the benefit of Japanese Patent Application No. 2011-228481 filed on Oct. 18, 2011, the disclosure of which is incorporated herein by reference.

2. Description of Related Art

Recently, an imaging device such as a digital still camera, a digital video camera, or the like that can acquire images captured by a sold-state imaging device (hereinafter referred to as an “image sensor”), as digital data and store and edit the digital data has become popular worldwide. As an image sensor used for the foregoing imaging device, a charge coupled device (CCD) type image sensor has been most widely used. Recently, however, as a demand for downsizing and low power consumption of the image sensor has increased, a complementary metal oxide semiconductor (CMOS) image sensor has been receiving attention and gaining popularity. Recently, with the increased demand for high speed and low power consumption, a CMOS type image sensor (a column ADC type CMOS image sensor) that includes AD conversion units installed to correspond to each pixel column and AD-converts pixel signals in each pixel column has been proposed.

A method for correcting linearity of input and output characteristics is described in Japanese Unexamined Patent Application, First Publication No. 2003-219172. In Japanese Unexamined Patent Application, First Publication No. 2003-219172, a solid-state imaging device includes a correction unit configured to divide a dynamic range of the AD conversion units into a plurality of regions, for each color of pixels provided in the solid-state imaging device and correct the linearity of the input and output characteristics in each region.

Hereinafter, the relationship between the dynamic range of the pixel signal and the correction of the linearity of the input and output characteristics will be described. FIG. 14A illustrates AD conversion characteristics of the AD conversion unit. In FIG. 14A, a horizontal axis represents analog voltage of the pixel signal input to the AD conversion unit and a vertical axis represents a value (an output value) of a digital signal output from the AD conversion unit. A curved line 1400 represents AD conversion characteristics of the AD conversion unit and a straight line 1410 represents ideal AD conversion characteristics. When the dynamic range of the pixel signal is general, the pixel signal in the overall range of the AD conversion characteristics illustrated in FIG. 14A is input to the AD conversion unit. Meanwhile, when the dynamic range of the pixel signal is narrow, the pixel signal in the narrower range than that of the AD conversion characteristics illustrated in FIG. 14A is input to the AD conversion unit. In FIG. 14A, when the dynamic range of the pixel signal is narrow, the dynamic range is about half of the general dynamic range. In Japanese Unexamined Patent Application, First Publication No. 2003-219172, a correction expression corresponding to the pixel signal of the general dynamic range is calculated and the digital signal output from the AD conversion unit is corrected based on the correction expression. The relationship between the pixel signal input to the AD conversion unit and the digital signal after being corrected substantially approximates the straight line 1410 and the linearity of the input and output characteristics is corrected.

FIG. 14B illustrates the AD conversion characteristics horizontally enlarged when the dynamic range of the pixel signal is narrow, among the AD conversion characteristics illustrated in FIG. 14A. In FIG. 14B, a horizontal axis represents analog voltage of the pixel signal input to the AD conversion unit and a vertical axis represents a value (an output value) of a digital signal output from the AD conversion unit. A curved line 1420 represents the AD conversion characteristics of the AD conversion unit when the dynamic range of the pixel signal is narrow. Further, a straight line 1430, which is a straight line of a portion corresponding to the case in which the dynamic range of the pixel signal is narrow, in the straight line 1410, represents the ideal AD conversion characteristics.

### SUMMARY OF THE INVENTION

According to a first aspect of the present invention, a solid-state imaging device includes: a pixel unit that has a plurality of pixels arranged in a matrix form, each of the plurality of pixels generating pixel signals, and outputs the pixel signals to a plurality of pixel signal output lines arranged to correspond to columns of the plurality of pixels; an AD conversion unit that is connected to one of the plurality of pixel signal output lines and converts the pixel signals output to the pixel signal output lines into digital signals to output the digital signals; a correction unit that corrects the digital signals output from the AD conversion unit based on a correction function using the digital signals output from the AD conversion unit as a variable so as to correct characteristics of the digital signals output from the AD conversion unit with respect to the pixel signals input to the AD conversion unit; and a correction method changing unit that changes an order of the variable between first and other orders in the correction function according to a change in a dynamic range of the pixel signal.

According to a second aspect of the present invention, in the solid-state imaging device according to the first aspect, the correction method changing unit changes the order of the variable in the correction function according to a change in the dynamic range of the pixel signal based on a photographing mode at the time of photographing.

According to a third aspect of the present invention, in the solid-state imaging device according to the first aspect, the correction method changing unit changes the order of the variable in the correction function according to a change in the dynamic range of the pixel signal based on a light source at the time of photographing.

According to a fourth aspect of the present invention, an endoscope device includes: the solid-state imaging device according to any one of the first to third aspects; a light source radiating illumination light to a subject; and a setting unit that changes a general observation mode setting the illumination light as general light and a special observation mode setting the illumination light as special light.

According to a fifth aspect of the present invention, a solid-state imaging device includes: a pixel unit that has a plurality of pixels arranged in a matrix form, each of the plurality of pixels detecting any one of multiple colors to generate pixel signals, and outputs the pixel signal to a plurality of pixel signal output lines arranged to correspond to the plurality of pixel columns; an AD conversion unit that is connected with one of the plurality of pixel signal output lines to convert and output the pixel signals output to the pixel signal output lines into digital signals; a correction function calculation unit that calculates a correction function for correcting characteristics of the digital signals output from the AD conversion unit with respect to the pixel signals input to the AD conversion unit based on the digital signals corresponding to the plurality of pixel signals corresponding to each of the multiple colors; and a correction unit that corrects the digital signal output from the AD conversion unit based on the correction function.

According to a sixth aspect of the present invention, in the solid-state imaging device according to the fifth aspect, the correction function calculation unit calculates the correction function based on the digital signals corresponding to the plurality of pixel signals corresponding to each of the multiple colors and a signal for adjusting white balance.

### BRIEF DESCRIPTION OF THE DRAWINGS

FIG. 1 is a block diagram illustrating a configuration of a solid-state imaging device according to a first embodiment of the present invention.

FIG. 2 is a flowchart illustrating an operation of the solid-state imaging device according to the first embodiment of the present invention.

FIG. 3A is a referential view for describing a method for obtaining a maximum value Vdmax and a minimum value Vdmin in a pixel signal of the first embodiment of the present invention.

FIG. 3B is a referential view for describing the method for obtaining the maximum value Vdmax and the minimum value Vdmin in the pixel signal of the first embodiment of the present invention.

FIG. 4 is a graph illustrating AD conversion characteristics for comparing the correction results of the related art to the correction results of the first embodiment of the present invention.

FIG. 5 is a graph illustrating the corrected results by the linear approximation correction of the related art.

FIG. 6 is a graph illustrating the results obtained by correcting linearity of input and output characteristics of a narrow D range mode by a method of the related art and the method of the first embodiment of the present invention.

FIG. 7 is a block diagram illustrating a configuration of a solid-state imaging device according to a second embodiment of the present invention.

FIG. 8 is a referential view illustrating a relationship between light source information and photographing modes in the second embodiment of the present invention.

FIG. 9 is a block diagram illustrating a configuration of an endoscope device according to a third embodiment of the present invention.

FIG. 10 is a block diagram illustrating a configuration of a solid-state imaging device according to a fourth embodiment of the present invention.

FIG. 11A is a referential view for describing a method for calculating a correction expression in the fourth embodiment of the present invention.

FIG. 11B is a referential view for describing the method for calculating the correction expression in the fourth embodiment of the present invention.

FIG. 11C is a referential view for describing the method for calculating the correction expression in the fourth embodiment of the present invention.

FIG. 12 is a block diagram illustrating a configuration of a solid-state imaging device according to the fourth embodiment (a modified example) of the present invention.

FIG. 13 is a block diagram illustrating a configuration of a solid-state imaging device according to a fifth embodiment of the present invention.

FIG. 14A is a graph for describing a problem in a correction of the related art.

FIG. 14B is a graph for describing a problem in a correction of the related art.

### DETAILED DESCRIPTION OF THE INVENTION

Hereinafter, embodiments of the present invention will be described with reference to the accompanying drawings.

### First Embodiment

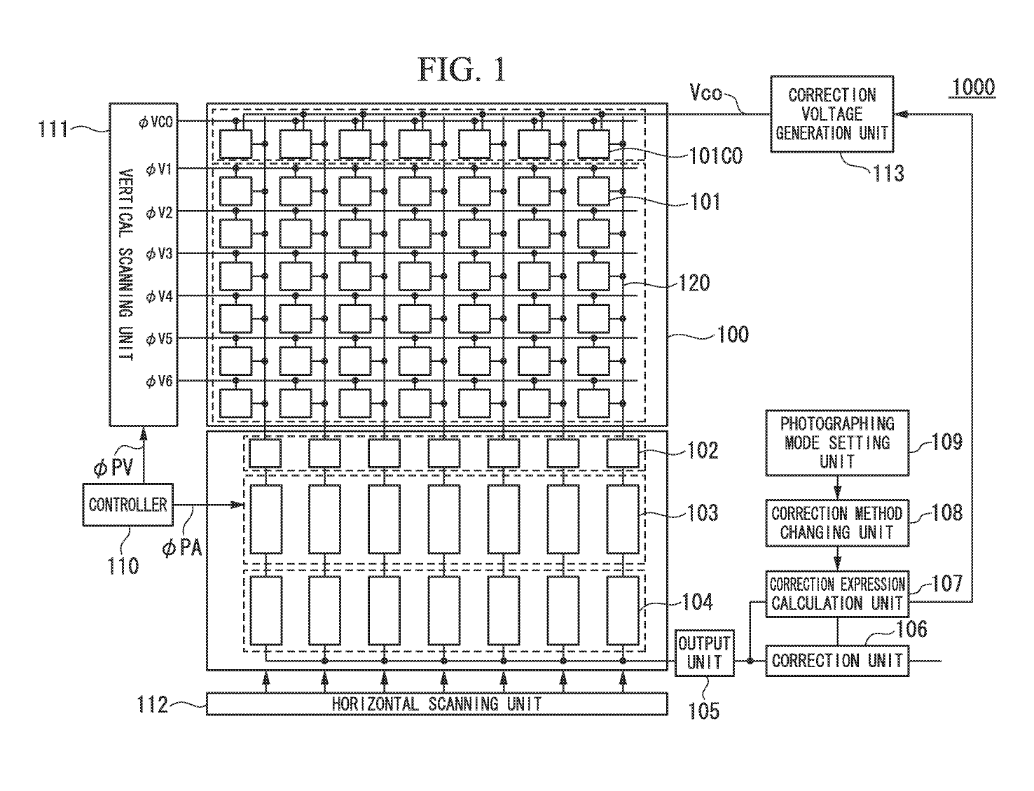

First, a first embodiment of the present invention will be described. FIG. 1 illustrates a configuration of a solid-state imaging device according to the present embodiment. Hereinafter, each component in FIG. 1 will be described. For simplification of explanation of each embodiment according to the present invention, the detailed configuration and operation of each component configuring the solid-state imaging device and a photographing operation of the solid-state imaging device is the same as the conventional art, and therefore the description thereof will be omitted.

Further, color filters are arranged in a pixel unit of the solid-state imaging device used for a digital still camera, or the like corresponding to colors, such as RGB, and a microcomputer or the like that is provided in a rear section of the solid-state imaging device performs image processing corresponding to each piece of color information. In the present embodiment, for simplification of explanation, color information of the pixel unit will be described as monochrome.

As illustrated in FIG. 1, a solid-state imaging device 1000 includes a pixel unit 100, an analog signal processing unit 102, an AD conversion unit 103, a memory unit 104, an output unit 105, a correction unit 106, a correction expression calculation unit 107, a correction method changing unit 108, a photographing mode setting unit 109, a controller 110, a vertical scanning unit 111, a horizontal scanning unit 112, and a correction voltage generation unit 113.

The pixel unit 100 includes a plurality of pixels 101 arranged in a matrix form and a plurality of correction pixels 101C0. In the present embodiment, the pixel 101 is described as being arranged in 7 columns and 6 rows. This is the same even in a second embodiment to be described below. Further, in the present embodiment, the correction pixels 101C0 corresponding to 1 row are arranged.

The pixel 101 generates a pixel signal based on an amount of incident light. A correction voltage Vco from the correction voltage generation unit 113 is applied to the correction pixel 101C0.

The correction pixel 101C0 outputs signals based on the correction voltage Vco from the correction voltage generation unit 113, regardless of the amount of incident light. Each pixel 101 and the correction pixels 101C0 are connected to the pixel signal output lines 120 that are arranged for each column (hereinafter indicated as pixel columns) configuring a pixel array. The pixel signals generated from each pixel 101 and the signals from the correction pixels 101C0 are output to the corresponding pixel signal output lines 120.

The vertical scanning unit 111 outputs various types of control signals to the pixel unit 100 to control an exposure operation or a signal reading operation of the pixel 101, a signal reading operation of the correction pixel 101C0, or the like. The control of the pixel 101 and the correction pixel 101C0 is performed in units of columns. That is, when the signal of the pixel 101 or the correction pixel 101C0 is read, the vertical scanning unit 111 selects a column that reads a signal and outputs a control signal to the pixel 101 or the correction pixel 101C0 of the selected column and outputs signals to the pixel signal output lines 120 from the pixel 101 or the correction pixel 101C0 of the selected column.

Further, the vertical scanning unit 111 is controlled by the controller 110 to perform an operation corresponding to a control signal φPV from the controller 110. The vertical scanning unit 111 generates control signals φVC0 and φV1 to φV6 based on the control signal φPV. The control signal φVC0 is output to the correction pixel 101C0 and the control signals φV1 to φV6 are output to first row to sixth row of the pixel 101. The correction pixel 101C0 is selected while the control signal φVC0 is in a high (H) level and the pixel 101 of the rows corresponding to the control signals φV1 to φV6 is selected while the control signals φV1 to φV6 are in a high (H) level.

The solid-state imaging device 1000 according to the present embodiment includes an analog signal processing unit 102, and AD conversion unit 103, and a memory unit 104, corresponding to each pixel column. In each pixel column, an analog signal from the pixel unit 100 is input to the analog signal processing unit 102.

The analog signal processing unit 102 performs correlated double sampling (CDS), sample and hold (S/H), or the like on the analog signal output from the pixel unit 100 and outputs the processed analog signal to the AD conversion unit 103. The AD conversion unit 103 is controlled by the controller 110 to AD-convert the input signal and output the AD-converted digital data (the digital signal) to the memory unit 104 while the control signal φPA from the controller 110 is in high (H). The memory unit 104 stores digital data that is the AD-converted result. The output unit 105 outputs the digital data stored in the memory unit 104 to the correction unit 106 and the correction expression calculation unit 107 arranged in the rear section of the solid-state imaging device.

The horizontal scanning unit 112 controls the reading of the digital data from the memory unit 104. The controller 110 outputs the control signals φPV and φPA to the vertical scanning unit 111 and the AD conversion unit 103 and controls the operation of the vertical scanning unit 111 and the AD conversion unit 103. The correction method changing unit 108 outputs the signal corresponding to the photographing mode set by the photographing mode setting unit 109 to the correction expression calculation unit 107 and controls the operation of the correction expression calculation unit 107.

The correction expression calculation unit 107 calculates the correction expression (a correction function) for correcting the signal output from the output unit 105, that is the digital data output from the AD conversion unit 103 corresponding to the signal output from the correction method changing unit 108, using an AD conversion result (digital data) of the pixel signal output from the pixel 101 and an AD conversion result (digital data) of the signals output from the correction pixel 101C0 and outputs the calculated correction expression to the correction unit 106. In this case, the pixel signal used to calculate the correction expression by the correction expression calculation unit 107 may be the pixel signal for calculating the correction expression that is acquired after power is applied to the solid-state imaging device or may be the pixel signal when a subject is actually photographed. Further, the correction expression calculation unit 107 determines the correction voltage Vco output by the correction voltage generation unit 113 based on the pixel signal and outputs a signal commanding the generation of the determined correction voltage Vco to the correction voltage generation unit 113.

The correction unit 106 corrects the digital data output from the output unit 105 using the correction expression calculated by the correction expression calculation unit 107 to perform the correction of the digital data so that the relationship between the analog voltage of the pixel signal and the corrected digital data is linear (straight). The correction voltage generation unit 113 is controlled by the correction expression calculation unit 107 to generate the correction voltage Vco based on the signal output from the correction expression calculation unit 107 and to output the generated correction voltage Vco to the correction pixel 101C0.

The photographing mode setting unit 109 outputs a signal according to the photographing mode selected by a photographer to the correction method changing unit 108. The photographing mode setting unit 109 includes an interface that is operated by a user and a photographer may operate the photographing mode setting unit 109 to select the photographing mode. The solid-state photographing device 1000 according to the present embodiment includes a normal mode used as a photographing state at a normal time as the photographing mode and a narrow D range mode that has the photographing state in which a dynamic range of the pixel signal is narrower than the normal mode.

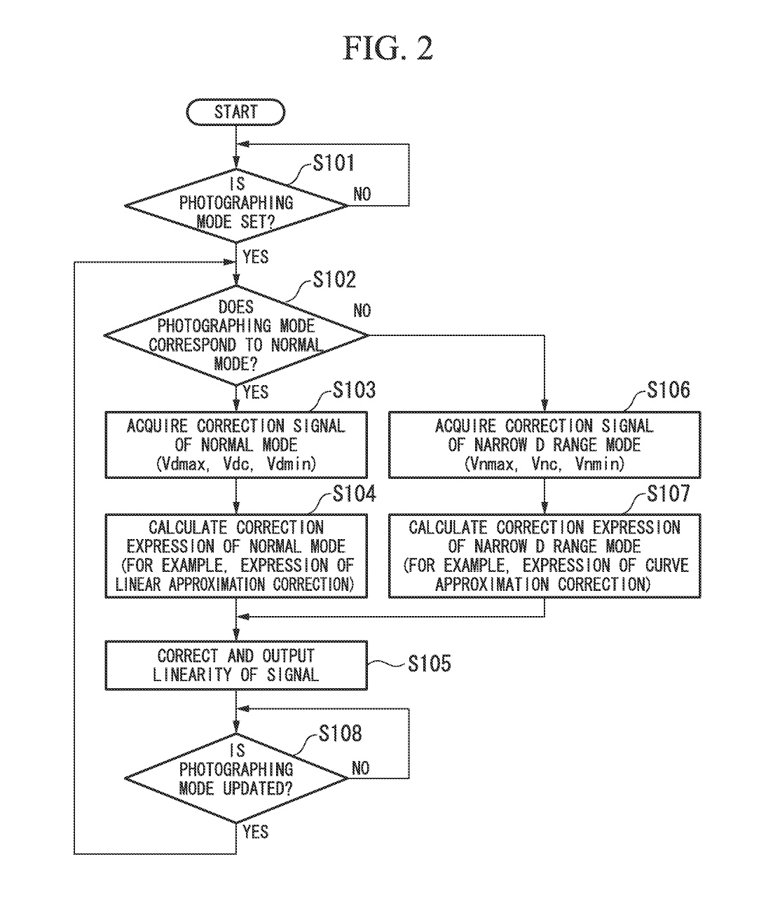

Next, the operation of the solid-state imaging device 1000 configured as described above will be described with reference to FIG. 2. FIG. 2 illustrates the operation of the solid-state imaging device 1000. When the solid-state imaging device 1000 receives power and starts, the operation illustrated in FIG. 2 is started. First, the correction method changing unit 108 determines whether the photographing mode is set by the photographing mode setting unit 109 (step S101). When the photographing mode is not set, a determination of step S101 is continued. Further, when the photographing mode is set, the correction method changing unit 108 determines whether the set photographing mode is a normal mode (step S102).

When the photographing mode set by the photographing mode setting unit 109 is a normal mode, the correction method changing unit 108 outputs a signal corresponding to the normal mode to the correction expression calculation unit 107. The correction expression calculation unit 107 calculates the correction expression appropriate for the normal mode based on the signal from the correction method changing unit 108 and outputs the calculated correction expression to the correction unit 106. The correction unit 106 corrects the digital data output from the output unit 105 using the correction expression and outputs the corrected digital data (steps S103 to S105).

Further, when the photographing mode set by the photographing mode setting unit 109 is the narrow D range mode, the correction method changing unit 108 outputs the signal corresponding to the narrow D range mode to the correction expression calculation unit 107. The correction expression calculation unit 107 calculates the correction expression appropriate for the narrow D range mode based on the signal from the correction method changing unit 108 and outputs the calculated correction expression to the correction unit 106. The correction unit 106 corrects the digital data output from the output unit 105 using the correction expression and outputs the corrected digital data (steps S106, S107, and S105).

After the digital data is corrected, the correction method changing unit 108 determines whether the photographing mode is updated by the photographing mode setting unit 109 (step S108). When the photographing mode is not updated, a determination of step S108 is continued. Further, when the photographing mode is updated, the process returns to step S102.

Next, steps S103 and S104 will be described in detail. First, the correction expression calculation unit 107 acquires the three-point correction signals for calculating the correction expression appropriate for the normal mode (step S103). Correction signals Vdmax and Vdmin of two of the three points are the digital data obtained by AD-converting a maximum value VDMAX and a minimum value VDMIN in the pixel signal obtained when an appropriate image is photographed in the normal mode. In addition, a correction signal Vdc of the remaining one of the three points is the digital data obtained by AD-converting the signals output from the correction pixel 101C0 to which the correction voltage Vco corresponding to any value in the range of the maximum value VDMAX and the minimum value VDMIN in the normal mode is input.

The correction voltage Vco for acquiring the correction signal Vdc may be a point of a region near a middle portion of the maximum value VDMAX and the minimum value VDMIN, a point of a region below the middle portion of the maximum value VDMAX and the minimum value VDMIN, or a point of a region above the middle portion of the maximum value VDMAX and the minimum value VDMIN and may be changed.

Next, the correction expression calculation unit 107 calculates the correction expression for correcting the digital data using each of the acquired correction signals (step S104). As the correction expression, the correction expression described in, for example, Japanese Unexamined Patent Application, First Publication No. 2004-274157 may be used. That is, the correction expression calculation unit 107 divides the range of the output value of the AD conversion unit 103 into a plurality of regions and obtains the correction expressions for each divided region.

In the present embodiment, the correction expression for dividing the range of the output value of the AD conversion unit 103 in two using the three correction signals is represented below.

Y1=A1×X+B1(Region 1:Vdmin≦X<Vdc) (1)

Y2=A2×X+B2(Region 2:Vdc≦X≦Vdmax) (2)

Here, X represents the value of the digital data before being corrected, Y1 and Y2 represent the values of the corrected digital data, A1 and A2 represent multiplier coefficients, and B1 and B2 represent intercepts of a linear function. The above Expression (1) and Expression (2) are correction expressions when the AD conversion characteristics of the AD conversion unit 103 are corrected (a linear approximation correction) to be approximate to a straight line and are linear functions using the digital data output from the AD conversion unit 103 as a variables. The calculation method of the above Expressions (1) and (2) is the same as the related art, and therefore the description thereof will be omitted.

For example, Expression (9) of Japanese Unexamined Patent Application, First Publication No. 2004-274157 may be used as the above Expressions (1) and (2).

Next, step S106 and step S107 will be described in detail. First, the correction expression calculation unit 107 acquires the three-point correction signals for calculating the correction expression appropriate for the narrow D range mode, as in the case of the normal mode. Correction signals Vnmax and Vnmin of two of three points are the digital data obtained by AD-converting a maximum value VNMAX and a minimum value VNMIN in the pixel signal obtained when an appropriate image is photographed in the narrow D range mode. In addition, a correction signal Vnc of the remaining one of the three points is the digital data obtained by AD-converting the signals output from the correction pixel 101C0 to which the correction voltage Vco corresponding to any value in the range of the maximum value VNMAX and the minimum value VNMIN in the narrow D range mode is input.

The correction voltage Vco for acquiring the correction signal Vnc may be a point of a region near a middle portion of the maximum value VNMAX and the minimum value VNMIN, a point of a region below the middle portion of the maximum value VNMAX and the minimum value VNMIN, or a point of a region above the middle portion of the maximum value VNMAX and the minimum value VNMIN and may be changed.

Next, the correction expression calculation unit 107 calculates the correction expression for correcting the digital data using each of the acquired correction signals (step S107). Here, the correction method is a method for correcting (a curve approximation correction) the AD conversion characteristics of the AD conversion unit 103 so as to be approximate to a curved line of a quadratic or higher order function, rather than the foregoing linear approximation correction. Even for the correction expression used for the correction, the correction expression of the related art may be used. For example, the correction expression is as follows.

Y=C1×X²+D1×X+E1(Vnmin≦X≦Vnmax) (3)

Here, X represents the value of the digital data before being corrected, Y represents the value of the corrected digital data, C1 and D1 represent multiplier coefficients, and E1 represents an intercept of a quadratic function. The above Expression (3) is the correction expression when the AD conversion characteristics of the AD conversion unit 103 are corrected (the curve approximation correction) to be approximate to a curved line of a quadratic function and is a quadratic function using the digital data output from the AD conversion unit 103 as a variable. The calculation method of the above Expression (3) is the same as the related art, and therefore the description thereof will be omitted. For example, Expression (15) of Japanese Unexamined Patent Application, First Publication No. 2004-274157 may be used as the above Expression (3).

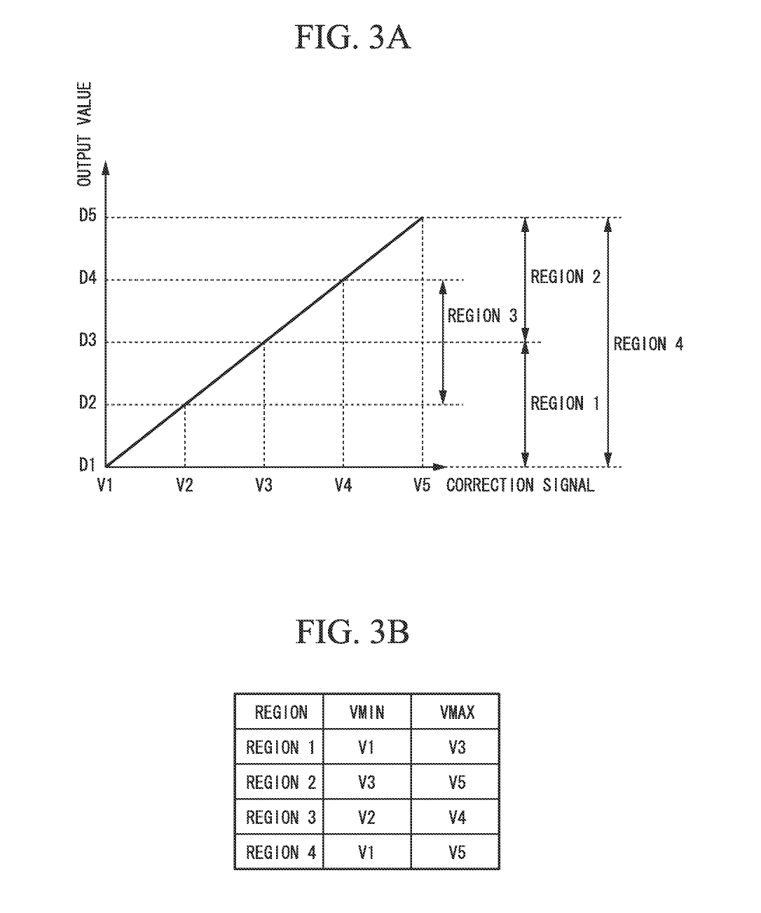

Next, referring to FIGS. 3A and 3B, when an appropriate image is photographed in a normal mode or a narrow D range mode, a method for obtaining a maximum value VMAX and a minimum value VMIN in the obtained pixel signal and a method for acquiring a correction signal will be described. FIG. 3A illustrates the AD conversion characteristics of the AD conversion unit 103. In FIG. 3A, a horizontal axis represents an analog voltage of the correction signal input to the AD conversion unit 103 and a vertical axis represents a value (an output value) of a digital signal output from the AD conversion unit 103. The AD conversion characteristics are divided into four regions (a region 1, a region 2, a region 3, and a region 4). The region 1 is a region in which the output value is from D1 to D3, the region 2 is a region in which the output value is from D3 to D5, the region 3 is a region in which the output value is from D2 to D4, and the region 4 is a region in which the output value is from D1 to D5 (D1<D2<D3<D4<D5). Each of the output values D1 to D5 corresponds to the corresponding analog voltage V1 to V5 of the correction signal. The values of the analog voltage V1 to V5 of the correction signal and each of the output values D1 to D5 are already known.

The correction expression calculation unit 107 detects a maximum value X1 and a minimum value X2 of the digital data output from the output unit 105 by the photographing of an image and determines whether the detected maximum value X1 and minimum value X2 are present in any of the region 1 to the region 4. The correction expression calculation unit 107 determines that the maximum value X1 and the minimum value X2 are present in the region 1 when the maximum value X1 and the minimum value X2 of the digital data have the relationship of D1≦X2<X1≦D3. Further, the correction expression calculation unit 107 determines that the maximum value X1 and the minimum value X2 are present in the region 2 when the maximum value X1 and the minimum value X2 of the digital data have the relationship of D3≦X2<X1≦D5. Further, the correction expression calculation unit 107 determines that the maximum value X1 and the minimum value X2 are present in the region 3 when the maximum value X1 and the minimum value X2 of the digital data have the relationship of D2≦X2<X1≦D4. Further, the correction expression calculation unit 107 determines that the maximum value X1 and the minimum value X2 are present in the region 4 when the maximum value X1 and the minimum value X2 of the digital data have the relationships of X2<D2 and D4<X1.

Next, the correction expression calculation unit 107 selects the maximum value VMAX and the minimum value VMIN of the pixel signal corresponding to the region in which the maximum value X1 and the minimum value X2 of the digital data are present. FIG. 3B illustrates the analog voltage at a boundary between respective regions and the relationship between the maximum value VMAX and the minimum value VMIN of the pixel signal. For example, the minimum value V1 of the analog voltage of the region 1 corresponds to the minimum value VMIN of the pixel signal and the maximum value V3 of the analog voltage of the region 1 corresponds to the maximum value VMAX of the pixel signal. Even in another region, the minimum value of the analog voltage corresponds to the minimum value VMIN of the pixel signal and the maximum value of the analog voltage corresponds to the maximum value VMAX of the pixel signal. That is, the analog voltage at a boundary of the region in which the maximum value X1 and the minimum value X2 of the digital data are present is considered as the maximum value VMAX and the minimum value VMIN of the pixel signal. The division of the region illustrated in FIGS. 3A and 3B is by way of example and it may obtain the maximum value VMAX and the minimum value VMIN of the pixel signal corresponding to the maximum value X1 and the minimum value X2 of the digital data with higher precision by dividing the region to be smaller.

Next, the correction expression calculation unit 107 selects any analog voltage VC between the maximum value VMAX and the minimum value VMIN of the pixel signal. The correction expression calculation unit 107 sequentially outputs the analog voltage VMAX, VMIN, and VC as the correction voltage Vco to the correction voltage generation unit 113. The correction expression calculation unit 107 acquires the correction signal output from the output unit 105 for each of the cases in which the correction voltage Vco is the analog voltage VMAX, the analog voltage VMIN, and the analog voltage VC. The correction signal corresponds to the correction signals Vdmax and Vnmax when the correction voltage Vco is the analog voltage VMAX, the correction signal corresponds to the correction signals Vdmin and Vnmin when the correction voltage Vco is the analog voltage VMIN, and the correction signal corresponds to the correction signals Vdc and Vnc when the correction voltage Vco is the analog voltage VC. As described above, the correction signals may be acquired.

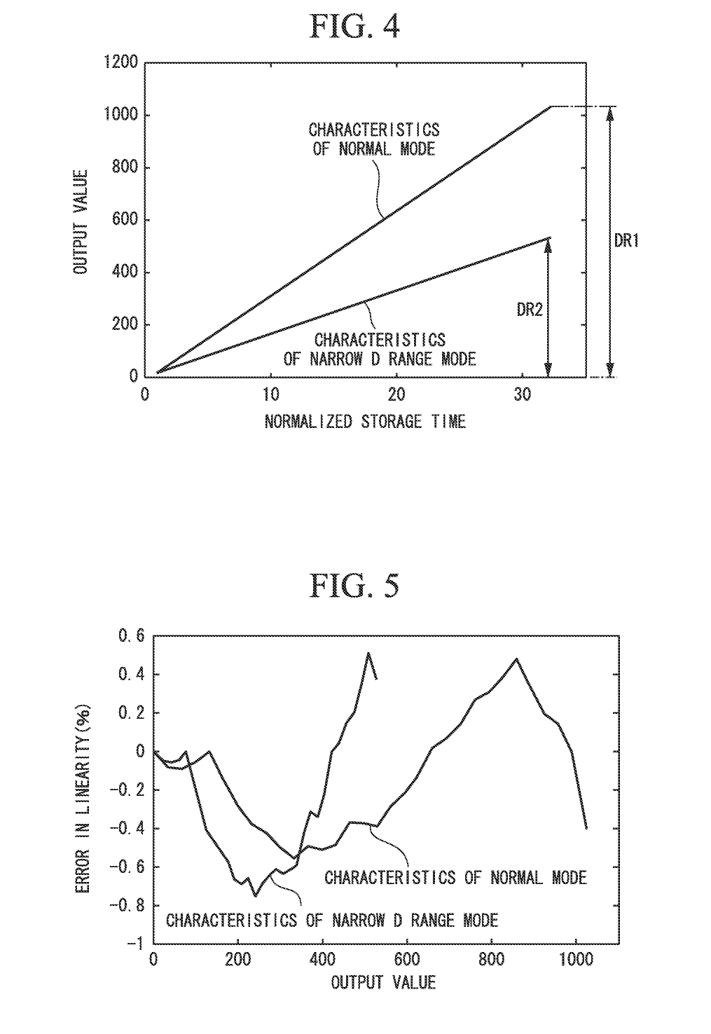

Next, the embodiment will be described with reference to FIGS. 4 to 6 by comparing the correction results by the linear approximation correction of the related art and the correction results by the linear approximation correction of the present embodiment. FIG. 4 illustrates the AD conversion characteristics of the AD conversion unit 103. In FIG. 4, a horizontal axis represents a storage time of the pixel 101 when the analog voltage of the pixel signal input to the AD conversion unit 103 is generated and a vertical axis represents the value (the output value) of the digital signal output from the AD conversion unit 103. In FIG. 4, the storage time of the horizontal axis is normalized in order to commonly represent the range of the pixel signal input to the AD conversion unit 103 in the normal mode and the narrow D range mode.

Here, the dynamic range of the signal is represented by a difference between the maximum value and the minimum value of the output value. DR1 becomes the dynamic range in the normal mode and DR2 becomes the dynamic range in the narrow D range mode. When the dynamic ranges of the normal node and the narrow D range mode are compared, it may be appreciated that DR2<DR1 and the AD conversion characteristics are changed by the photographing mode.

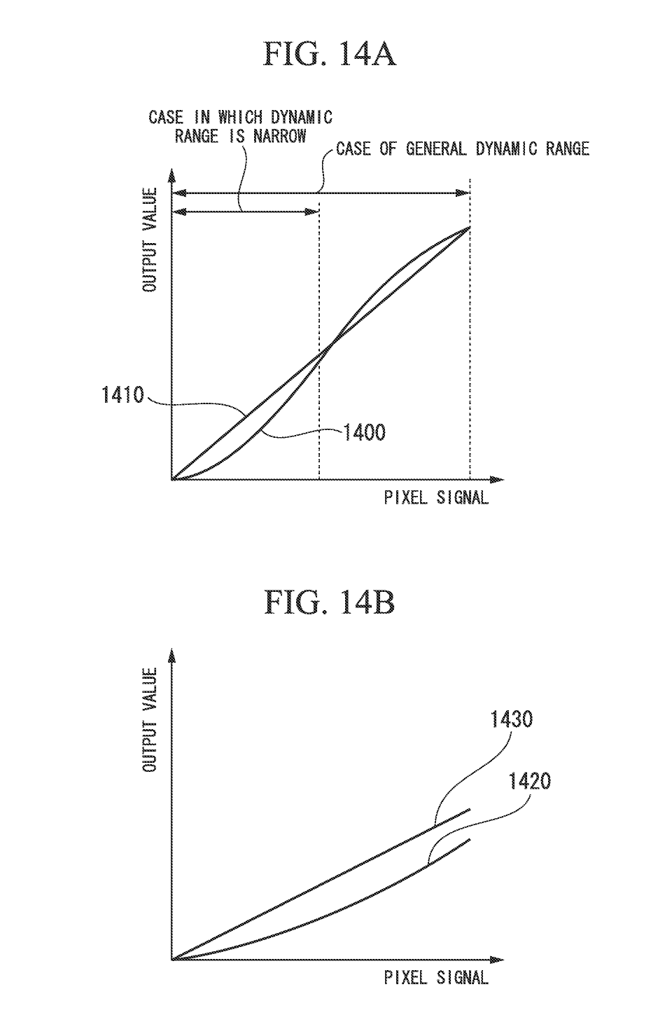

Hereinafter, the characteristics representing the relationship between the pixel signal input to the AD conversion unit 103 and the corrected digital data are referred to as the input and output characteristics. FIG. 5 illustrates the corrected results by the linear approximation correction of the related art. In FIG. 5, a horizontal axis represents the corrected output value and a vertical axis represents errors (errors in linearity) from the ideal input and output characteristics (corresponding to straight lines 1410 and 1430 of FIGS. 14A and 14B) in the normal mode. The correction method uses the linear approximation correction that may correct the linearity of the input and output characteristics with high precision.

When the errors in linearity between the input and output characteristics of the normal mode and the input and output characteristics of the narrow D range mode are compared, the error in linearity of the input and output characteristics of the normal mode is −0.56% to +0.49%, while the error in linearity of the input and output characteristics of the narrow D range mode is −0.76% to +0.52%. From this, it may be appreciated that the errors in linearity of the input and output characteristics of the narrow D range mode are larger. This is due to the following reason. When the photographing mode is changed from the normal mode to the narrow D range mode, the dynamic range of the pixel signal input to the AD conversion unit 103 is changed and the range of the AD conversion characteristics used in the AD conversion unit 103 is changed. Therefore, as described with reference to FIGS. 14A and 14B, when the linearity of the input and output characteristics of the narrow D range mode is corrected by the linear approximation correction using the correction expression calculated from the input and output characteristics of the normal mode, the correction cannot be sufficiently performed.

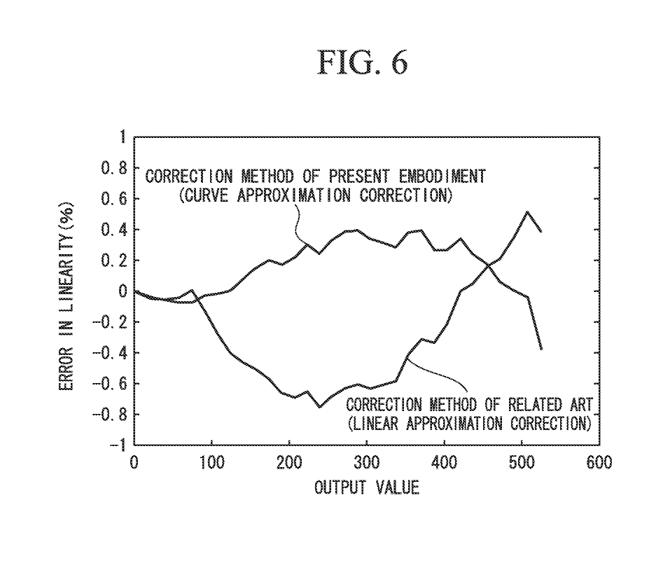

FIG. 6 illustrates the results obtained by correcting the linearity of the input and output characteristics of the narrow D range mode of FIG. 4 by a method of the related art and the method of the present embodiment. In FIG. 6, a horizontal axis represents the corrected output value and a vertical axis represents errors (errors in linearity) from the ideal input and output characteristics (corresponding to straight lines 1410 and 1430 of FIGS. 14A and 14B) in the normal mode. The results obtained by performing the correction by the correction method (the linear approximation correction) of the related art are the same as the results obtained by correcting the linearity of the input and output characteristics of the narrow D range mode illustrated in FIG. 5. The correction method of the present embodiment is the curve approximation correction that corrects the input and output characteristics of the narrow D range mode so as to be approximate to the curved line of the quadratic function. When the errors in linearity between the linear approximation correction and the curve approximation correction are compared, as described above, the error in linearity of the linear approximation correction is −0.76% to +0.52%, while the error in linearity of the curve approximation correction is −0.38% to +0.39%, such that the error in linearity of the curve approximation correction is improved.

As described above, according to the present embodiment, the linearity of the input and output characteristics is corrected by changing the correction method of the linearity of the input and output characteristics to the correction method appropriate for the set photographing mode, corresponding to the photographing mode set by the photographing mode setting unit 109. Therefore, even when the dynamic range of the input signal is changed according to the photographing mode, the linearity of the input and output characteristics may be corrected with higher precision.

Although the present embodiment describes the case in which the number of correction signals used in order to correct the linearity is set to be three points, the correction signal may be set to be at least three points, and may increase to four points or more. In this case, the correction expression is complicated due to the increase in the number of correction signals, and thus the increase in an operation load for calculating the correction expression, or the like, is considered, but the linearity of the input and output characteristics may be corrected with higher precision. When the number of correction signals is set to be four points or more, the curve approximation correction is performed by a cubic curve when the correction signal is set to be four points and is performed by a quartic curve when the correction signal is set to be five points, such that the correction signal is increased and an order of the correction expression of the curve approximation correction is also increased.

Further, the present embodiment describes the correction method appropriate for the normal mode as the linear approximation correction and the correction method appropriate for the narrow D range mode as the curve approximation correction, but is not limited thereto. The correction method optimal for the photographing mode may be set by confirming the pixel and the characteristics of the AD conversion unit in advance, in the manufacturing process or the initial setting process of the solid-state imaging device.

Further, the present embodiment describes the method for using the pixel signal of the photographed image and the signal of the correction pixel as the method for acquiring the correction signal, but is not limited thereto. As a first example of another method for acquiring the correction signal, a method for outputting the correction voltage corresponding to each correction signal of all the points in the correction voltage generation unit and acquiring the correction voltage as the correction signal may be used.

In addition, the same effect may be obtained using a method for acquiring an output signal of a reference voltage generation unit input to the analog signal processing unit or the AD conversion unit as the correction signal based on the configuration in which the output signal of the reference voltage generation unit is input to the analog signal processing unit or the AD conversion unit, as a second example of another method of acquiring the correction signal.

Further, the present embodiment describes the color information of the pixel unit as monochrome, but the pixel unit may be configured to obtain color information of three colors by general RGB color filters and the pixel unit may be configured to obtain color information of four colors or more. In this case, the same effect may be obtained by performing the processing of the present embodiment on each color.

### Second Embodiment

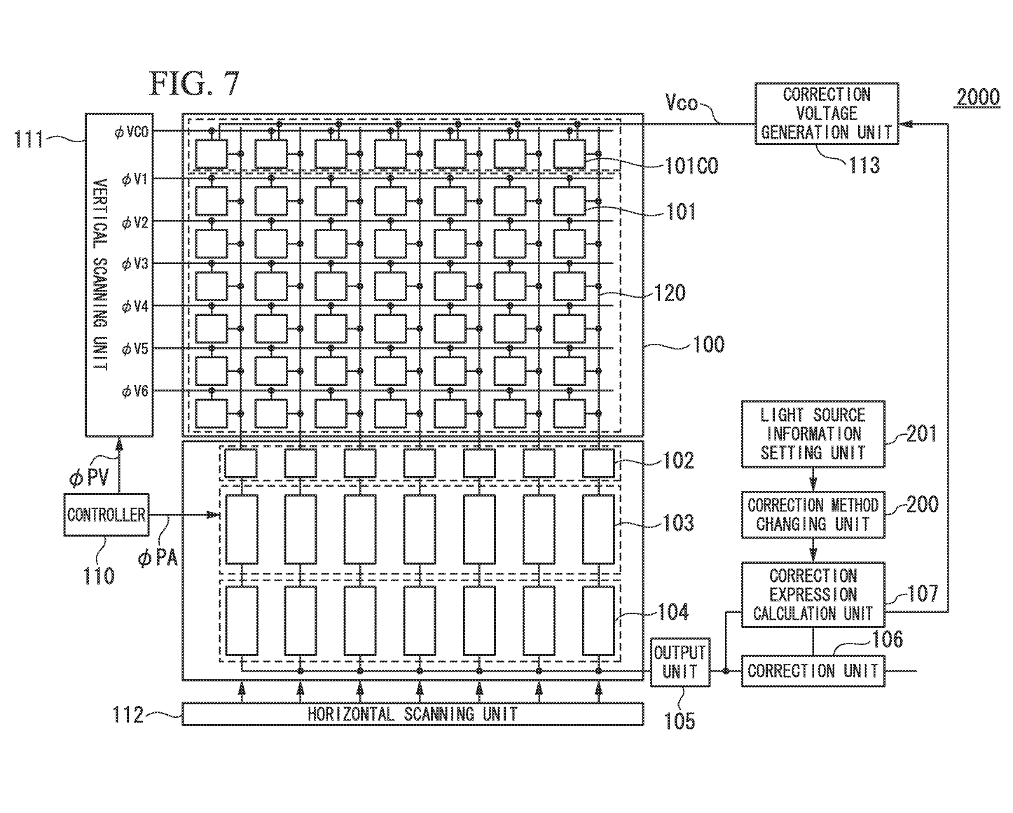

Next, a second embodiment of the present invention will be described. FIG. 7 illustrates a configuration of a solid-state imaging device according to the present embodiment. Among the components used in FIG. 7, the same components as the components used in FIG. 1 are denoted by the same reference numerals and the description thereof will be omitted. Hereinafter, each component and operation of the present embodiment will be described with a focus on different components from the first embodiment.

The difference between the present embodiment and the first embodiment is that the correction method changing unit 108 is changed to a correction method changing unit 200 and the photographing mode setting unit 109 is changed to a light source information setting unit 201. The light source information setting unit 201 includes an interface that can be operated by a user, and a photographer can set light source information such as a kind of a light source, light quantity, spectral sensitivity, or the like, by operating the light source information setting unit 201. The light source information setting unit 201 outputs a signal according to the light source information set by the photographer to the correction method changing unit 200.

The correction method changing unit 200 determines whether the light source information is set to correspond to the normal mode or set to correspond to the narrow D range mode according to the light source information set by the light source information setting unit 201 and outputs a signal corresponding to the determined result to the correction expression calculation unit 107. Hereinafter, the relationship between the light source information and the photographing mode will be described.

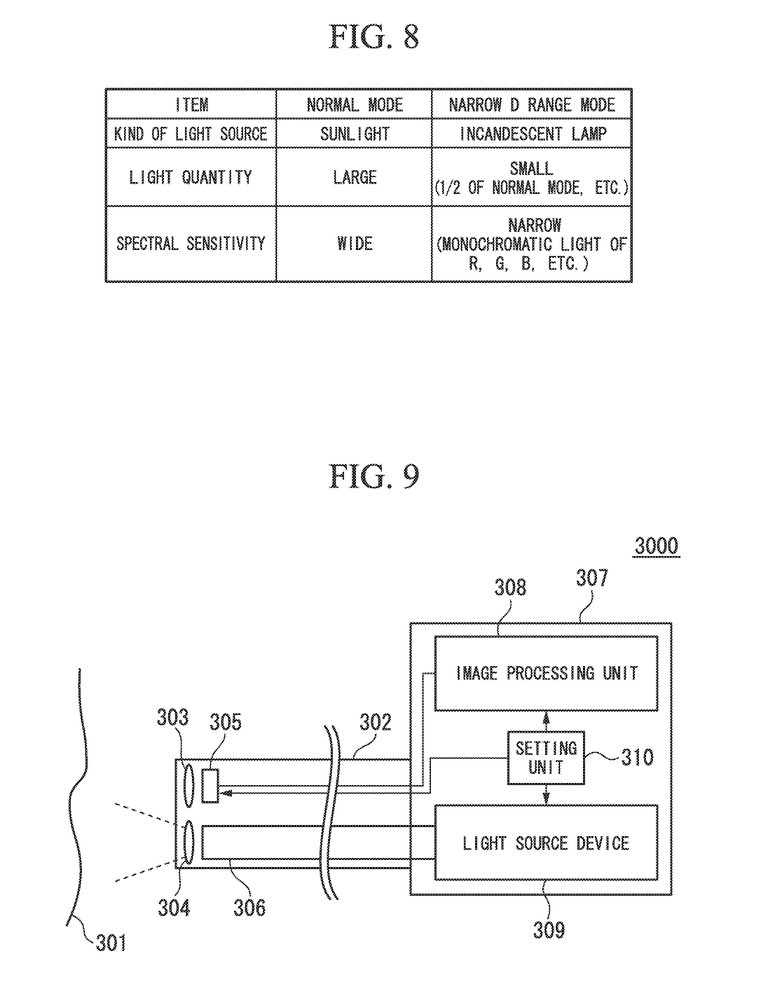

FIG. 8 illustrates the relationship between the light source information and the photographing mode. When the kind of the light source is sunlight, the photographing mode is the normal mode and when the kind of the light source is an incandescent lamp, the photographing mode is the narrow D range mode. Further, when the light quantity is large, the photographing mode is the normal mode and when the light quantity is small, the photographing mode is the narrow D range mode. Further, when the spectral sensitivity is wide, the photographing mode is the normal mode, and when the spectral sensitivity is narrow, the photographing mode is the narrow D range mode. The correction method changing unit 200 determines whether the light source information is set to correspond to the photographing mode of any one of the normal mode and the narrow D range mode according to the relationship illustrated in FIG. 8. In the present embodiment, the operation of the linearity correction is the same as the operation described in the first embodiment, and therefore the description thereof will be omitted.

As described above, according to the present embodiment, the linearity of the input and output characteristics is corrected by changing the correction method of the linearity of the input and output characteristics to the correction method appropriate for the light source information according to the light source information set by the light source information setting unit 201. Therefore, even when the dynamic range of the input signal is changed according to the kind of the light source or light quantity, the linearity of the input and output characteristics can be corrected with higher precision.

Although the present embodiment describes that the photographer sets the light source information, a sensor detecting the kind of the light source or a sensor detecting the light quantity of the light source are further installed and the light source information may be automatically set based on the output signals of these sensors.

### Third Embodiment

Next, a third embodiment of the present invention will be described. The third embodiment describes, by way of example, an endoscope device as the specific utility form in the case of using the solid-state imaging device according to the present invention. FIG. 9 illustrates a configuration of an endoscope device including the solid-state imaging device according to the present embodiment. Hereinafter, each component in FIG. 9 will be described.

As illustrated in FIG. 9, an endoscope device 3000 includes a scope 302 and an enclosure 307. The scope 302 includes a solid-state imaging device 305 that is an applied example of the present invention, a lens 303 focusing reflected light from a subject on the solid-state imaging device 305, a fiber 306 transmitting an illumination light to a subject, and a lens 304 radiating the illumination light to the subject. Further, the enclosure 307 includes a light source device 309 including a light source that generates the illumination light radiated to a subject, an image processing unit 308 that performs predetermined processing on a signal output from the solid-state imaging device 305 to generate a photographed image, and a setting unit 310 that sets the photographing (observation) mode of the endoscope device.

The foregoing endoscope device 3000 includes, as the photographing mode, a general observation mode in which the normal illumination light is used as the illumination light radiated to the subject and a special observation mode in which a state of a blood vessel, or the like, is obtained for a depth direction in the vicinity of a mucous membrane outer layer easily buried in the optical information obtained in the general observation mode. An example of a document in which the special observation mode is described may include, for example, Japanese Patent No. 3586157.

The normal mode of the foregoing first and second embodiments corresponds to the general observation mode and the narrow D range mode of the foregoing first and second embodiments corresponds to the special observation mode.

When the general observation mode is set as the photographing mode by the setting unit 310, the signal corresponding to the general observation mode is output to the solid-state imaging device 305, the image processing unit 308, and the light source device 309 from the setting unit 310 and the observation is performed by the general illumination light. In addition, when the special observation mode is set as the photographing mode by the setting unit 310, the signal corresponding to the special observation mode is output to the solid-state imaging device 305, the image processing unit 308, and the light source device 309 from the setting unit 310 and the observation is performed by illumination light in a different band than usual.

The specific example of the special observation mode may include narrow band imaging (NBI) in which observation is performed using the light source of the narrow band. In order to detect a blood vessel with high contrast, the NBI uses blue light (390 nm to 445 nm) and green light (530 nm to 550 nm) that is obtained by forming white light into a narrow band by an optical filter, rather than general white light, as the light source of the radiated light. Therefore, the quantity of light incident on the solid-state imaging device is reduced and amplitude of the pixel signal of the solid-state imaging device 305 is small, such that the dynamic range is narrow. As a result, when the linearity of the input and output characteristics of the solid-state imaging device 305 is corrected as in the general observation mode, the linearity may not be sufficiently corrected. However, the linearity of the input and output characteristics can be corrected with higher precision by using the solid-state imaging device described in the first and second embodiments and the image quality of the photographed image of the endoscope device may be improved.

### Fourth Embodiment

Next, a fourth embodiment of the present invention will be described. For simplification of explanation, the foregoing first and second embodiments describe the case in which the color information of the pixel unit is monochrome. However, the color filters for multiple colors are arranged in the pixel unit of the solid-state imaging device according to the utility form of the solid-state imaging device. Generally, for example, in the digital still camera, the RGB color filters are provided with a pixel unit having an R pixel, a G pixel, and a B pixel arranged in an order that is referred to as a Bayer array and the color image of the subject may be obtained by performing the image processing corresponding to each piece of color information obtained from the R pixel, the G pixel, and the B pixel. The Bayer array corresponds to one in which the RGB color filters are arranged in a matrix, wherein the R and G color filters or the B and G color filters are arranged in each pixel column.

When the foregoing RGB color filters are arranged, the dynamic ranges in the pixels of each color are different. Therefore, there is a need to calculate each correction expression which corrects the linearity of the input and output characteristics for the pixels of each color, as disclosed in Japanese Unexamined Patent Application, First Publication No. 2003-219172. Further, when being considered in a column ADC type image sensor including the AD conversion units for each pixel column, there is a need to perform the correction of the linearity for the pixel signals of two colors for each AD conversion unit of each pixel column. Therefore, the operation load for calculating the correction expression is increased twofold, as compared with the case of monochrome.

Further, in the correction processing of the linearity, there is a need to perform the processing by sequentially changing the correction expressions to the correction expressions corresponding to each color according to an order reading the pixel signals from the pixels of each color, and as a result, the correction processing is complicated. Therefore, the present embodiment is to provide the solid-state imaging device that may more easily perform the linearity correction processing of the input and output characteristics.

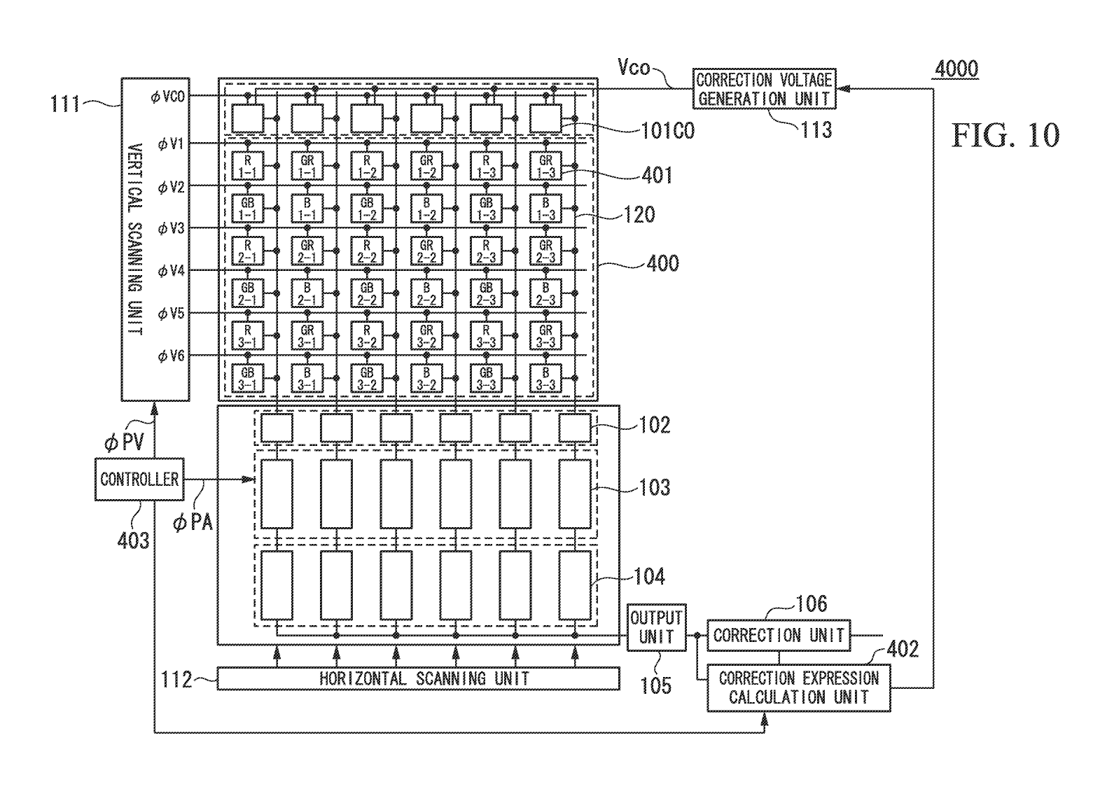

FIG. 10 illustrates a configuration of the solid-state imaging device according to the present embodiment. Among the components used in FIG. 10, the same components as the components used in FIG. 1 are denoted by the same reference numerals and the description thereof will be omitted. Hereinafter, each component and operation of the present embodiment will be described with a focus on different components from the first and second embodiments.

The difference between the present embodiment and the first and second embodiments is that the pixel unit 100 is changed to a pixel unit 400, the correction expression calculation unit 107 is changed to a correction expression calculation unit 402, and the controller 110 is changed to a controller 403. The pixel unit 400 includes a plurality of pixels 401 arranged in a matrix form and the plurality of correction pixels 101C0. In the present embodiment, the pixel 401 is described as being arranged in 6 columns and 6 rows. This is the same even in a fifth embodiment to be described below. Further, in the present embodiment, the correction pixels 101C0 corresponding to 1 row are arranged.

The pixel 401 generates the pixel signal corresponding to the RGB color filters (not shown) based on the amount of the incident light. R*-* (“*” is one of 1 to 3) of the pixel 401 is the R pixel, B*-* is the B pixel, and GR*-* and GB*-* are the G pixels. “*-*” is affixed to a character representing colors of each pixel to represent a column number and a row number, wherein a first figure represents a column number and a last number represents a row number. For example, “R2-3” represents an R pixel that is arranged in second row and third column.

The correction voltage Vco from the correction voltage generation unit 113 is applied to the correction pixel 101C0. The correction pixel 101C0 outputs signals based on the correction voltage Vco from the correction voltage generation unit 113 regardless of the amount of the incident light. Each pixel 401 and the correction pixel 101C0 are connected to the pixel signal output lines 120 that are arranged for each pixel column. The pixel signals generated from each pixel 401 and the signals from the correction pixels 101C0 are output to the corresponding pixel signal output lines 120.

The vertical scanning unit 111 is controlled by the controller 403 to perform an operation corresponding to the control signal φPV from the controller 403. The controller 403 outputs the control signals φPV and φPA to the vertical scanning unit 111 and the AD conversion unit 103, and controls the operation of the vertical scanning unit 111 and the AD conversion unit 103.

The correction expression calculation unit 402 calculates the correction expression using the digital data obtained by AD-converting the pixel signals output from the pixel 401 and the digital data obtained by AD-converting the signals output from the correction pixel 101C0. In more detail, the correction expression calculation unit 402 acquires R pixel correction signals VRmin, VRc, and VRmax and G pixel correction signals VGRmin, VGRc, and VGRmax from the digital data of the pixel signals for each first, third and fifth columns of the pixel unit 400 in which the R pixel and the G pixel are arranged, and calculates the correction expressions for each column using the R pixel correction signals VRmin, VRc, and VRmax and the G pixel correction signals VGRmin, VGRc, and VGRmax. Further, the correction expression calculation unit 402 generates B pixel correction signals VBmin, VBc, and VBmax and G pixel correction signals VGBmin, VGBc, and VGBmax from the digital data of the pixel signals for each second, fourth and sixth columns of the pixel unit 400 in which the B pixel and the G pixel are arranged, and calculates the correction expressions for each column using the B pixel correction signals VBmin, VBc, and VBmax and the G pixel correction signals VGBmin, VGBc, and VGBmax. That is, one correction expression corresponding to each pixel column is calculated.

The method for acquiring each of the R pixel correction signals VRmin, VRc, and VRmax, the G pixel correction signals VGRmin, VGRc, and VGRmax, the G pixel correction signals VGBmin, VGBc, and VGBmax, and the B pixel correction signals VBmin, VBc, and VBmax is the same as the method for acquiring three-point correction signals in the first embodiment. According to the first embodiment, for example, the R pixel correction signals VRmin and VRmax correspond to the correction signals Vdmax, Vnmax, Vdmin, and Vnmin corresponding to the maximum value VMAX and the minimum value VMIN in the pixel signal obtained when an appropriate image is photographed. Further, according to the first embodiment, the R pixel correction signal VRc corresponds to the correction signals Vdc and Vnc corresponding to any value within the range of the maximum value VMAX and the minimum value VMIN. This is likewise applied to the correction signals of the pixels of other colors.

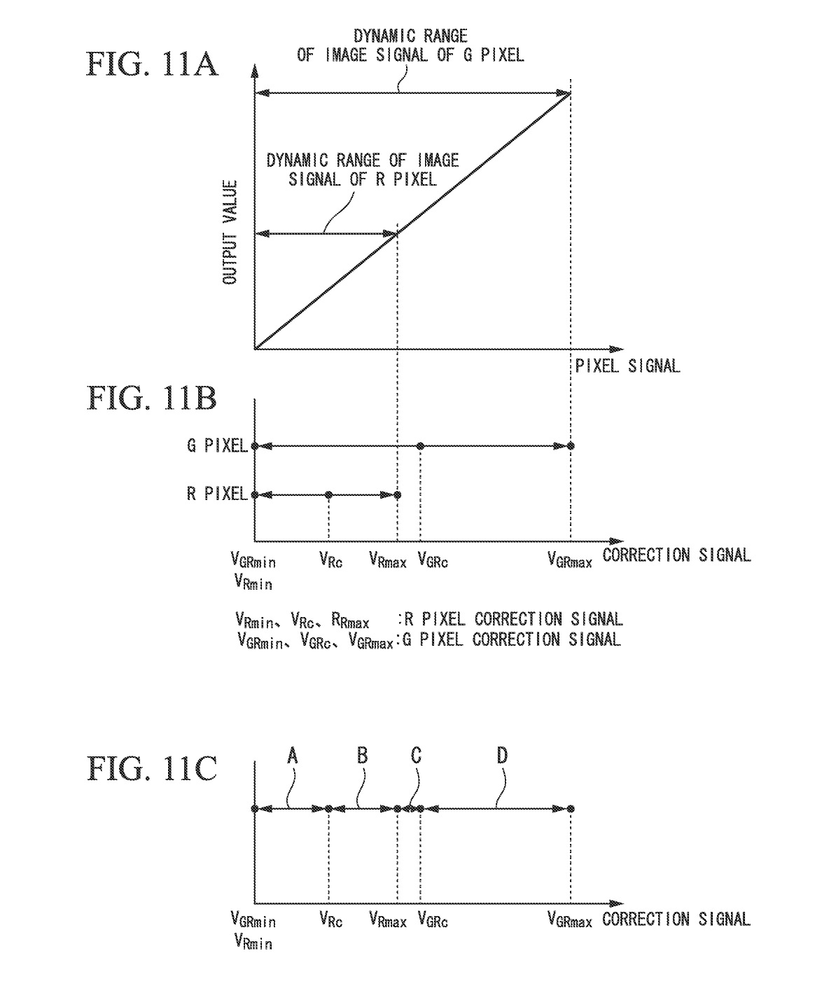

Hereinafter, each of correction signals and the correction expressions will be described with reference to FIGS. 11A to 11C based on, for example, the R pixel correction signals VRmin, VRc, and VRmax and the G pixel correction signals VGRmin, VGRc, and VGRmax. FIG. 11A illustrates the AD conversion characteristics of the AD conversion unit 103 when the pixel signal of the G pixel has a dynamic range wider than that of the R pixel, for the pixel signals of the G pixel and the R pixel when the image is photographed. In FIG. 11A, a horizontal axis represents the analog voltage of the pixel signal input to the AD conversion unit 103 and a vertical axis represents the value (the output value) of the digital signal output from the AD conversion unit 103. FIG. 11B illustrates the relationship between the dynamic range of the pixel 401 and the correction signal. In FIG. 11B, the horizontal axis represents the digital value of the correction signal. For simplification of explanation, the dynamic range of the pixel signal coincides with the range in which the digital value of the correction signal is included.

The dynamic range of the pixel signal of the R pixel is narrower than that of the G pixel and therefore, the amplitude of the pixel signal of the R pixel is narrower than that of the G pixel. In FIG. 11A, the amplitude of the pixel signal of the R pixel is about 40% of that of the pixel signal of the G pixel. Therefore, in order to correct the linearity of the input and output characteristics corresponding to the pixel signals of each pixel, the optimal correction signal is different in the G pixel and the R pixel. For the R pixel, the R pixel correction signals VRmin, VRc, and VRmax become the optimal correction signals and for the G pixel, the G pixel correction signals VGRmin, VGRc, and VGRmax become the optimal correction signals. In order to correct the linearity of the input and output characteristics corresponding to the pixel signal of the B pixel when considering the R pixel and the G pixel as the same, the optimal correction signals are the B pixel correction signals VBmin, VBc, and VBmax corresponding to the dynamic range of the pixel signal of the B pixel.

The correction expression calculation unit 402 calculates each correction expression of first, third and fifth columns of the pixel unit 400 in which the R pixel and the G pixel are arranged based on the R pixel correction signals VRmin, VRc, and VRmax, and the G pixel correction signals VGRmin, VGRc, and VGRmax. In more detail, the range of the output value of the AD conversion unit 103 is divided into four regions based on the R pixel correction signals VRmin, VRc, and VRmax and the G pixel correction signals VGRmin, VGRc, and VGRmax, and thus the correction expressions corresponding to each region are calculated. FIG. 11C illustrates the four regions. The four regions correspond to a region A between the R pixel correction signal VRmin or the G pixel correction signal VGRmin and the R pixel correction signal VRc, a region B between the R pixel correction signal VRc and the R pixel correction signal VRmax, a region C between the R pixel correction signal VRmax and the G pixel correction signal VGRc, and a region D between the G pixel correction signal VGRc and the G pixel correction signal VGRmax. In each of the regions A to D, the correction expressions of each regions are calculated using the correction pixel signals at the boundary of the each regions.

As described above, the correction expression calculation unit 402 calculates each correction expression of second, fourth and sixth columns of the pixel unit 400 in which the B pixel and the G pixel are arranged based on the B pixel correction signals VBmin, VBc, and VBmax, and the G pixel correction signals VGBmin, VGBc, and VGBmax. As described above, one correction expression for each pixel column is calculated. The correction expression calculation unit 402 outputs the calculated correction expressions of each pixel column to the correction unit 106. The correction unit 106 corrects the digital data output from the output unit 105 using the correction expression calculated by the correction expression calculation unit 402.

As described above, according to the present embodiment, the correction expression correcting the linearity of the input and output characteristics for each color is not separately calculated based on the correction signals corresponding to colors (in the present embodiment, R, and B) of each pixel, but the correction expression correcting the linearity of the input and output characteristics corresponding to multiple colors (in the present embodiment, R and G, or B and G) is calculated based on the correction signals corresponding to colors of each pixel, such that the operation load for calculating the correction expression may be reduced. For example, in the present embodiment, two kinds of correction expressions each corresponding to two colors for each pixel column are not calculated, but for each pixel column, one kind of correction expression corresponding to both of the two colors is calculated, such that the operation load may be reduced.

Alternatively, when the correction expressions for each color are separately calculated, the correction expressions corresponding to two regions divided by setting the analog voltage of, for example, the maximum value, the minimum value, and the intermediate value as the boundary are calculated. However, unlike this, in the present embodiment, the correction expressions for each of four regions (the regions A to D of FIGS. 11A to 11C) divided by setting the analog voltage of the maximum value, the minimum value, and the intermediate value corresponding to each of the plurality of colors as the boundary are calculated. Therefore, the correction expression that approximates the ideal linearity of the input and output characteristics with higher precision may be calculated. In this way, the linearity of the input and output characteristics may be corrected with higher precision.

Further, the correction processing does not change the correction expression according to the change in colors of pixels reading the pixel signals, but is performed to change the correction expression according to the change in the pixel column reading the pixel signal. Therefore, it may lower the frequency of changing the correction expression and facilitate the correction processing.

Further, the present embodiment describes the method for using the pixel signal of the photographed image and the signal of the correction pixel as the method for acquiring the correction signal, but is not limited thereto. As a first example of another method for acquiring the correction signal, a method for outputting the correction voltage corresponding to each correction signal of all the points in the correction voltage generation unit and acquiring the correction voltage as the correction signal may be used.

In addition, the same effect may be obtained using a method for acquiring the output signal of the reference signal generation unit input to the analog signal processing unit or the AD conversion unit as the correction signal based on the configuration in which the output signal of the reference voltage generation unit is input to the analog signal processing unit or the AD conversion unit, as a second example of another method of acquiring the correction signal.

Although the present embodiment describes the case in which the number of correction signals used in order to correct the linearity is set to be three points, the correction signal may be set to be at least three points, and may be increased to four points or more. In this case, the correction expression is complicated due to the increase in the number of correction signals, and thus the increase in an operation load for calculating the correction expression, or the like, is considered, but the linearity of the input and output characteristics may be corrected with higher precision.

Next, a modified example according to the present embodiment will be described. In the present embodiment the AD conversion units are disposed in each pixel column and the correction expressions for each pixel column (for each pair of the R pixel and the G pixel and for each pair of the B pixel and the G pixel) are calculated, but the present embodiment is not limited thereto. The single correction expression corresponding to each color of RGB may be calculated based on all the correction signals corresponding to each color of RGB.

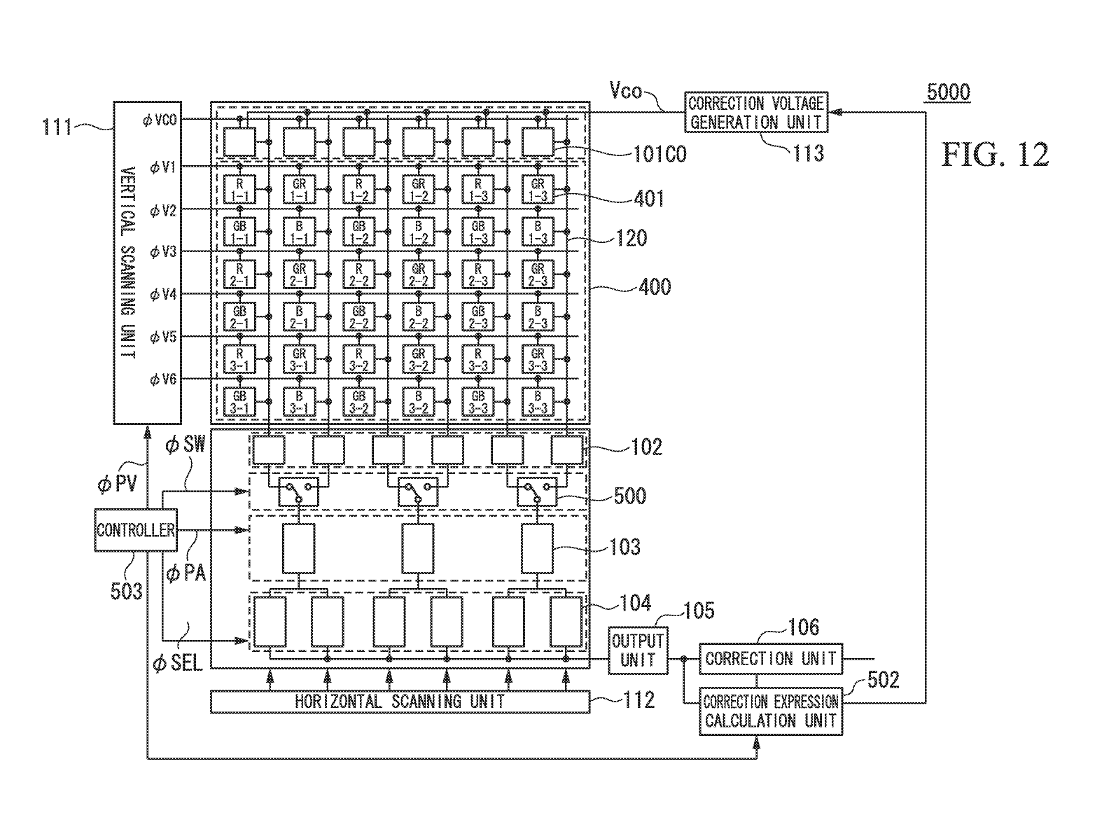

FIG. 12 illustrates an example of a specific configuration of a solid-state imaging device according to the present modified example. Among the components used in FIG. 12, the same components as the components used in FIG. 1 are denoted by the same reference numerals and the description thereof will be omitted. A solid-state imaging device 5000 illustrated in FIG. 12 includes the AD conversion units 103 that are arranged at a ratio of one to two columns and a changing unit 500 that changes the pixel signals input to the AD conversion units 103 between the pixel signals of two columns. The pixel signals of two columns correspond to one AD conversion unit 103 and the pixel signals are input to the AD conversion unit 103 while changing the pixel signals of two columns. The AD conversion unit 103 outputs the AD-converted digital data to the memory units 104 of to two columns.

The horizontal scanning unit 112 selects the memory unit 104 of the pixel column corresponding to the pixel signal that is input to the AD conversion unit 103 by the changing unit 500 and outputs the digital data. For example, when the pixel signal of the left pixel column among the left and right pixel columns is input to the AD conversion unit 103, the digital data is output from the memory unit 104 of the left pixel column, and when the pixel signal of the right pixel column among the left and right pixel columns is input to the AD conversion unit 103, the digital data is output from the memory unit 104 of the right pixel column.

In this configuration, the single AD conversion unit AD-converts the pixel signal of RGB of two columns and therefore each AD conversion unit may generate the correction signals corresponding to the RGB of two columns and calculate the single correction expression correcting the digital data of two columns using these correction signals. The detailed configuration or operation of FIG. 12 will be omitted.

### Fifth Embodiment

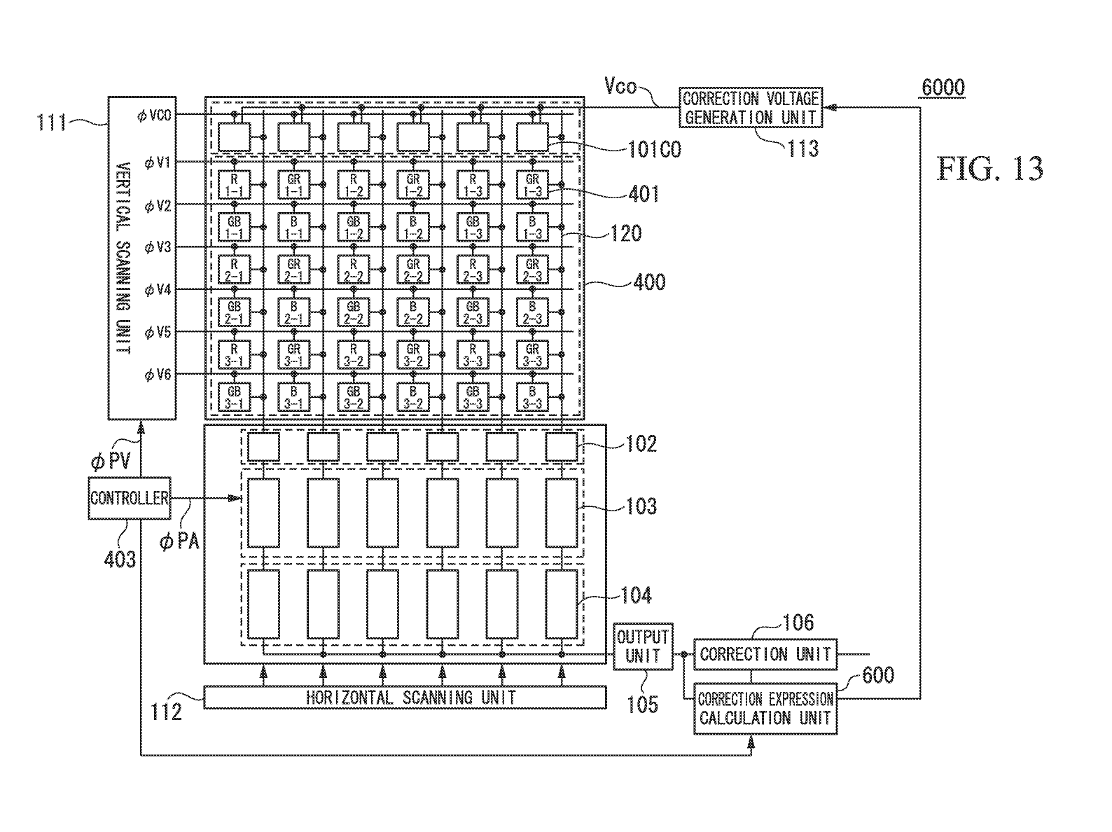

Next, a fifth embodiment of the present invention will be described. FIG. 13 illustrates a configuration of a solid-state imaging device according to the present embodiment. Among the components used in FIG. 13, the same components as the components used in FIG. 10 are denoted by the same reference numerals and the description thereof will be omitted. Hereinafter, the components and operations of the present embodiment will be described with a focus on different components from the fourth embodiment.

The difference between the present embodiment and the fourth embodiment is that the correction expression calculation unit 402 is changed to a correction expression calculation unit 600. The correction expression calculation unit 600 calculates the correction expression using signals VWR and VWB for adjusting white balance, in addition to the correction signal based on the pixel signals of each pixel. The signals VWR and VWB are digital signals for adjusting the white balance of the R pixel and B pixel, respectively. Even when the color temperature of the light source is changed at the time of photographing, the white balance means general processing of performing accurate white correction by adjusting the white signal of the photographed image.

The correction expression calculation unit 600 calculates each correction expression of first, third and fifth columns of the pixel unit 400 in which the R pixel and the G pixel are arranged using the R pixel correction signals VRmin, VRc, and VRmax, the G pixel correction signals VGRmin, VGRc, and VGRmax, and the signal VWR. In more detail, based on the same concept as the fourth embodiment, the range of the output value of the AD conversion unit 103 is divided into four regions based on the R pixel correction signals VRmin, VRc, and VRmax the G pixel correction signals VGRmin, VGRc, and VGRmax, and the signal VWR into four regions, and thus the correction expressions corresponding to each region are calculated. Similarly, the correction expression calculation unit 600 calculates each correction expression of second, fourth and sixth columns of the pixel unit 400 in which the B pixel and the G pixel are arranged using the B pixel correction signals VBmin, VBc, and VBmax, the G pixel correction signals VGBmin, VGBc, and VGBmax, and the signal VWB. The correction expression calculation unit 600 outputs the calculated correction expressions of each pixel column to the correction unit 106. The correction unit 106 corrects the digital data output from the output unit 105 using the correction expression calculated by the correction expression calculation unit 600.

According to the present embodiment, the order of the variable is changed between first and other orders in the correction function according to the dynamic range of the pixel signal, and thus, even when the dynamic range of the pixel signal is changed, the characteristics of the digital signal output from the AD conversion unit with respect to the pixel signal input to the AD conversion unit may be corrected with higher precision.

In the present embodiment, it may minimize errors in linearity at an adjustment point using the adjustment point of the white balance as the correction signal while keeping the characteristics minimizing the errors in linearity at each point of the correction signal using the calculation of the general correction expression. In addition, it may improve the image quality of the solid-state imaging device by suppressing the coloring (the color shift) affecting the errors in linearity.

While preferred embodiments of the present invention have been described, the present invention is not limited to the embodiments. Additions, omissions, substitutions, and other variations may be made to the present invention without departing from the spirit and scope of the present invention. The present invention is not limited by the above description, but by the appended claims.

What is claimed is:

1. A solid-state imaging device, comprising:
a pixel unit that has a plurality of pixels arranged in a matrix form, each of the plurality of pixels generating pixel signals, and outputs the pixel signals to a plurality of pixel signal output lines arranged to correspond to columns of the plurality of pixels;
an AD conversion unit that is connected to one of the plurality of pixel signal output lines and converts the pixel signals output to the pixel signal output lines into digital signals to output the digital signals;
a correction unit that corrects the digital signals output from the AD conversion unit based on a correction function using the digital signals output from the AD conversion unit as a variable so as to correct characteristics of the digital signals output from the AD conversion unit with respect to the pixel signals input to the AD conversion unit; and
a correction method changing unit that changes an order of the variable between first and other orders in the correction function according to a change in a dynamic range of the pixel signals.

2. The solid-state imaging device according to claim 1, wherein the correction method changing unit changes the order of the variable in the correction function according to a change in the dynamic range of the pixel signals based on a photographing mode at the time of photographing.

3. The solid-state imaging device according to claim 1, wherein the correction method changing unit changes the order of the variable in the correction function according to a change in the dynamic range of the pixel signals based on a light source at the time of photographing.

4. An endoscope device, comprising:
the solid-state imaging device according to any one of claims 1 to 3; a light source that radiates illumination light to a subject; and
a setting unit that changes a general observation mode setting the illumination light as general light and a special observation mode setting the illumination light as special light.
